(19) United States
(12) Patent Application Publication
(10) Pub. No.: US 2016/0249805 A1
(43) Pub. Date: Sep. 1, 2016

(54) AUXILIARY FLASH ASSEMBLY FOR A MEDICAL INSTRUMENT

(71) Applicant: Welch Allyn, Inc., Skaneateles Falls, NY (US)

(72) Inventor: Jon R. Salvati, Skanealeles, NY (US)

(21) Appl. No.: 14/634,009

(22) Filed: Feb. 27, 2015

(51) Int. Cl. A61B 3/14; A61B 3/12; A61B 5/00; G03B 15/02

(52) U.S. Cl. CPC A61B 3/14; G03B 15/02; A61B 3/12; A61B 5/6898

(57) ABSTRACT

An auxiliary flash apparatus for a medical diagnostic instrument having an attached portable electronic device. The auxiliary flash apparatus includes a light detector and a trigger circuit associated with the light detector. The light detector upon receipt of illumination output from the source of flash illumination creates a trigger pulse that is received by signal conditioning electronics connected to the trigger circuit. The signal conditioning electronics are configured to produce a synchronized and re-created flash via at least one connected light source of the medical diagnostic instrument. The re-created flash output can be synchronized or substantially synchronized therewith.

### TECHNICAL FIELD

This application is generally directed to the field of diagnostic medicine, and more specifically to a medical instrument having an attached portable electronic device, such as a smart-phone or similar type of mobile communications device, the latter including an integrated electronic imager and flash assembly. When coupled, the output of the flash assembly can be synchronously or substantially synchronously re-created by an illumination assembly of the medical instrument when the flash assembly is fired.

### BACKGROUND

Diagnostic instruments, including various hand-held versions, are routinely utilized in the medical field for purposes of examining a patient. Examples of medical diagnostic instruments can include laryngoscopes, vagiscopes, otoscopes, ophthalmoscopes, skin surface microscopes, rhinoscopes, and anoscopes, among others. These instruments can include both optical, as well as digital or electronic versions, the latter versions typically incorporating an integrated electronic imager that is aligned with a contained optical assembly and configured to receive images of a specific medical target(s) of interest (e.g., the eyes, ear, nose, throat, skin, etc.). In both optical and digital instrument versions, a contained light source is also typically provided in order to adequately illuminate the target of interest and permit imaging/viewing.

More recently, Applicants have developed a medical diagnostic instrument (i.e., an ophthalmoscope) commercially sold under the iExaminer® trademark. Rather than employing a dedicated and integral electronic imager, the instrument includes an “off the shelf” portable electronic device (i.e., a smart-phone such as an Apple iPhone®) that is releasably retained within a holding fixture/adapter engaged at the proximal end of an existing instrument housing. The holding fixture/adapter is defined by an open-ended cavity, which is appropriately sized and configured to engage the lateral surfaces of the smartphone and enable placement thereof in a predetermined orientation. In this orientation, the camera lens of the smart-phone is aligned with the optical assembly of the diagnostic instrument and configured to receive images using the smart-phone's resident display. When assembled, the display of the smart-phone is accessible to the user, including the ability to capture images using the instrument via the smart-phone's resident software and user interface, including camera functions. Regardless of the operating system (e.g., Google™ Android®, Apple iOS), the portable electronic device is already configured to fire its internal flash assembly whenever an image is captured and in instances in which ambient lighting conditions are not deemed favorable. However and when combined with imaging-enabled medical devices, the optical assembly of the medical diagnostic instrument may partially or completely block the flash output of the attached portable electronic device. Even if the flash output is not blocked and depending upon the intended target of interest, the flash output of the smart-phone or other connected device may still not be of sufficient intensity or may possess a color temperature that is not appropriate or optimal for purposes of conducting an effective medical examination.

### BRIEF DESCRIPTION

Therefore and according to one aspect, there is provided an auxiliary flash apparatus for a medical diagnostic instrument having an attached portable electronic device, the portable electronic device having an electronic imaging device and a source of flash illumination. A light detector is configured to receive a flash output from the source of flash illumination and a trigger circuit is associated with the light detector. Signal conditioning electronics are associated with the trigger circuit for receiving a trigger pulse from the trigger circuit synchronously or substantially synchronously with the flash output. The signal conditioning electronics is associated with a light source of the medical diagnostic instrument wherein the signal conditioning electronics are configured to produce a substantially synchronized and re-created flash output using the light source.

In at least one embodiment, the electronic device can be a smart-phone and in which the light source comprises at least one LED.

The signal conditioning electronics may comprise a pulse shaping and amplifier circuit wherein the light detector can comprise a photocell.

According to another aspect, there is provided a medical instrument comprising a device housing having a light source and a portable electronic device that is coupled to the device housing, the portable electronic device including an electronic imager and a flash assembly. A light detector is configured to detect a flash output of the portable electronic device and a trigger circuit is associated with the light detector. Signal conditioning electronics are associated with the trigger circuit for receiving a trigger pulse from the trigger circuit synchronously or substantially synchronously with the flash output and the signal conditioning electronics are associated with a light source of the medical instrument, wherein the signal conditioning electronics is further configured to produce a substantially synchronized and re-created flash using the light source.

In one version, the portable electronic device is a smart-phone, although other mobile communications devices such as tablet computers, PDAs, digital camcorders and the like can similarly be utilized. The medical diagnostic instrument can be an ophthalmoscope for examining the eyes according to at least one version, although the herein described apparatus can be utilized for literally any medical diagnostic instrument typically configured for examining a medical target of interest and having a separately attached portable electronic device.

The light detector in at least one version can include a photocell, wherein the light source can include at least one LED. The signal conditioning electronics may comprise a pulse shaping and amplifier circuit.

According to yet another aspect, there is provided a method for synchronously re-creating a flash of a portable electronic device attached or otherwise engaged with a medical instrument, the medical instrument including a light detector disposed in relation to a flash assembly of the attached portable electronic device. According to the method and when an image is captured by the portable electronic device, light emitted by the flash assembly of the portable electronic device is detected by a light sensing apparatus. The light sensing apparatus converts the light emitted by the flash assembly into an electrical signal. The electrical signal is then directed to a trigger circuit, as well as to signal conditioning electronics, which may include a pulse shaper and amplifier circuit either synchronously or substantially synchronously with the flash output. One or more light sources of the medical instrument, are configured to produce a synchronized or substantially synchronized flash output. In at least one version, the flash output produced by the one or more light sources of the instrument can have adequate light intensity, dispersion and color temperature that are best suited for the intended medical imaging application.

The portable electronic device can be a smart-phone according to at least one described embodiment, although other mobile communication devices such as tablet computers, PDAs, digital camcorders and the like can similarly be utilized that includes an integrated camera and flash assembly. The medical diagnostic instrument can be an ophthalmoscope for examining the eyes but it should be noted that this selection is entirely exemplary. That is, the form of instrument can be varied to any hand-held or sufficiently portable device configured for examining a medical target of interest such as an anoscope, otoscope, laryngoscope, skin surface microscope, vagiscope or other instrument that further includes a separately attached image capturing device having a source of flash illumination.

The light detector in at least one version can include a photocell wherein the one or more light sources can include at least one LED or an LED array. The signal conditioning electronics may optionally comprise a pulse shaping and amplifier circuit.

Advantageously, a flash can be effectively and synchronously or substantially synchronously re-created from the attached portable electronic device using the illumination assembly of the medical instrument and without having to specifically configure or position the portable electronic device to enable flash. Otherwise, an effective utilization of the existing flash of the portable electronic imaging device would require the use of various structures including but not limited to mirrors, light guides and/or beam splitters in order to adequately direct the flash output to the intended medical target for imaging purposes. These various structures would add significant cost and complexity to the instrument, even assuming that the flash output (e.g., intensity and/or color temperature) is adequate for the intended target of interest.

In addition, the flash output of the portable electronic device can be used for timing purposes in order to re-create the flash in a manner that is either more or less intense than the original flash output. Furthermore, the re-created flash output may also include a different color temperature.

Still further, the herein described instrument and auxiliary flash apparatus can further enable the use of aiming LEDs, allowing the medical instrument to have pre-flash illumination capability for the purpose of guiding the instrument into a dark cavity, such as an ear canal or relative to the eye of a patient, such as when using an eye cup.

These and other features and advantages will be readily apparent from the following Detailed Description, which should be read in conjunction with the accompanying drawings.

### BRIEF DESCRIPTION OF THE DRAWINGS

FIG. 1 is a side perspective view of a known medical diagnostic instrument in accordance with the prior art;

FIG. 2 is a rear perspective view of the medical diagnostic instrument of FIG. 1;

FIG. 3 is a cross sectional view of a medical diagnostic instrument including contained imaging and illumination systems;

FIG. 4 is a functional diagram of an auxiliary flash apparatus for use with a medical diagnostic instrument having an attached portable electronic device;

FIG. 5 is an exemplary schematic electrical circuit diagram of the auxiliary flash apparatus of FIG. 4;

FIG. 6 is a graphical representation demonstrating the response time for an exemplary light detector to produce a trigger pulse; and

FIG. 7 is a side elevational view, in section, of a portion of another exemplary medical instrument having an auxiliary flash apparatus.

### DETAILED DESCRIPTION

The following description relates to exemplary embodiments of an auxiliary flash apparatus that can be used in conjunction with a medical diagnostic instrument in order to effectively and synchronously or substantially synchronously re-create a flash output from that of an attached portable electronic device, such as a mobile communications device (e.g., a smart-phone or tablet computer). The herein described apparatus is functionally configured to utilize the output of a source of flash illumination of the attached portable electronic device and synchronously (or substantially synchronously) re-create an effective flash output in order to effectively image/view a medical target of interest, such as, for example, the nose, ears, eyes, etc., for purposes of conducting a diagnostic evaluation of a patient. The exemplary embodiments described herein relates to specific (ophthalmic and otoscopic) devices, but it will be readily apparent to one of sufficient skill that numerous variations and modifications embodying the inventive aspects can be contemplated, including use in connection with other medical diagnostic instruments capable of having electronic imaging devices used therewith. In addition and throughout the course of the following discussion, several terms are used in order to provide a suitable frame of reference in regard to the accompanying drawings. These terms, which include “upper”, “lower, “distal”, “proximal”, “above”, “below”, “front”, “behind” and the like are not intended to specifically limit the inventive concepts as described and claimed, except where so specifically indicated.

It should further be noted that the drawings herein provided are solely intended to illustrate the salient features of exemplary medical instruments having an auxiliary flash and related methods. Therefore, these drawings are not necessarily drawn to scale and should not be relied upon in terms of specific sizing of any depicted components or features.

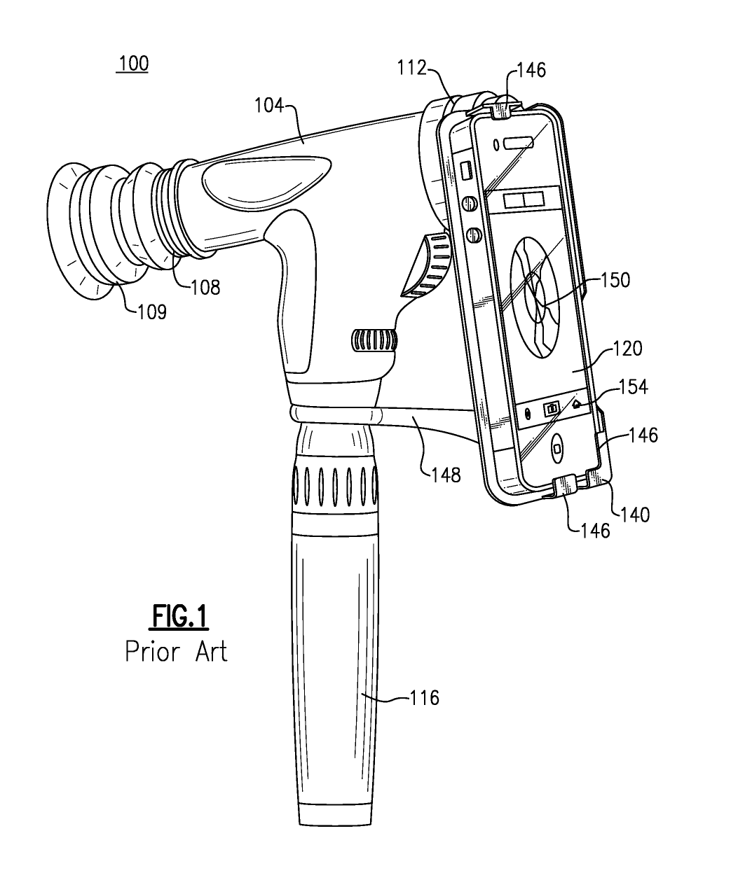

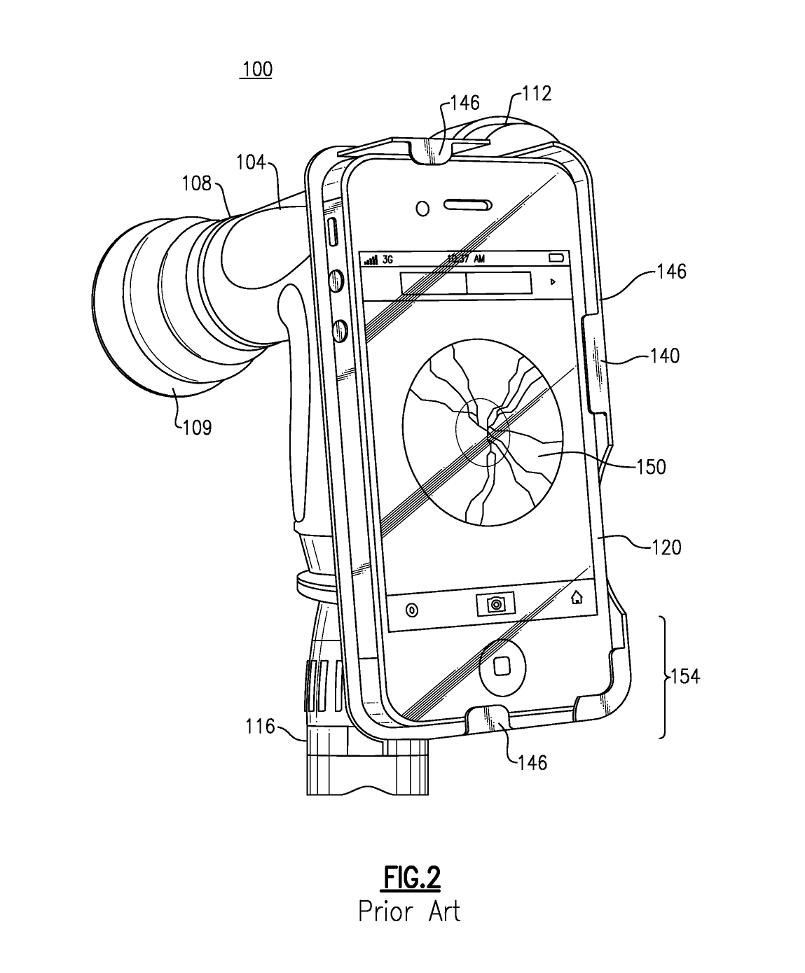

Referring first to FIGS. 1 and 2 and for purposes of background, a known medical diagnostic instrument 100 is herein described. This known medical instrument 100 is a portable (i.e., hand-held) ophthalmoscope configured for examining the eyes of a patient (not shown). The depicted instrument 100 is defined by a housing 104 having a distal end 108, as well as an opposing proximal end 112. A flexible eye cup 109 is releasably attached to the distal end 108 of the instrument 100, the eye cup 109 serving as a patient interface. As noted previously, the specific diagnostic instrument 100 that is described herein is exemplary and provided for illustrative purposes in order to provide a suitable background.

A downwardly extending section of the instrument housing 104 defines a handle portion 116 enabling the device 100 to be hand-held by a user (not shown) with an upper portion of the housing 104 defining an instrument head that is further defined by a hollow interior. The interior of the instrument head is appropriately configured and sized in order to retain a plurality of components and in which the handle portion 116 is similarly sized and configured to retain a set of rechargeable batteries (not shown) serving as a power source.

A portable electronic device 120, i.e., a smart-phone, is releasably attached to an adapter 140 that is provided at the proximal end 112 of the instrument housing 104. The adapter 140 is defined by an open-ended bracket or frame that includes a set of peripheral (side) rails 146 that enable sliding or snapfitting attachment, the adapter 140 being sized to fully retain the portable electronic device 120, while permitting access to the display 150 and user interface 154 of the device 120 when attached, including the camera functions thereof. The adapter 140 can include a lower support 148 extending to the handle 116 of the instrument 100 to provide adequate stability in supporting the portable electronic device 120. When attached, the camera lenses of the attached portable device are aligned with the imaging system of the medical instrument. Details relating to exemplary adapters are provided in U.S. Patent Application Publication No. 2012/0320340 and U.S. Patent Application Publication No. 2013/0083185, the entire contents of which are herein incorporated by reference.

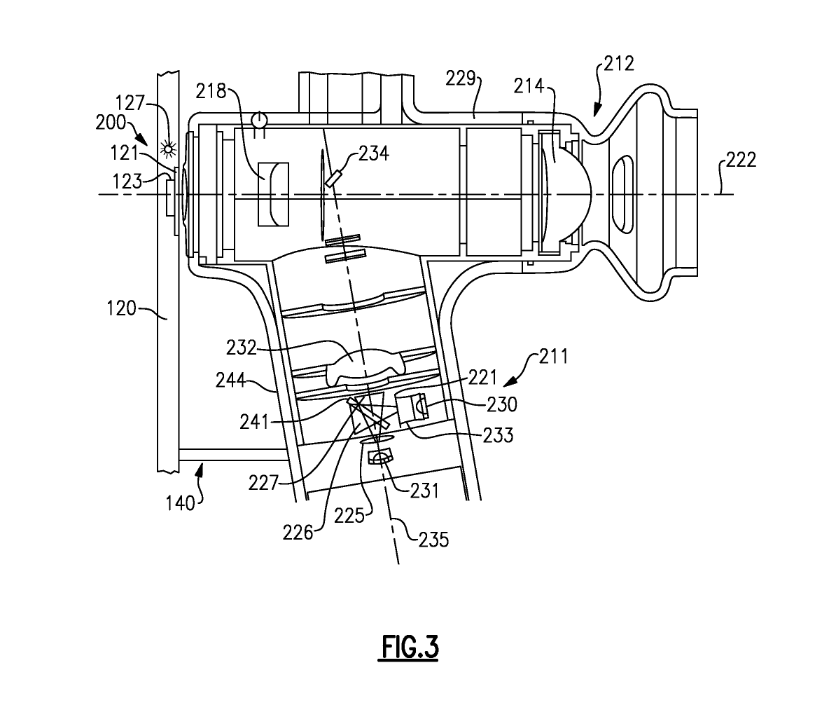

Referring to FIG. 3 and for purposes of additional background, a sectioned view of another exemplary medical diagnostic instrument 200 is depicted. The medical instrument 200 depicted herein is also an ophthalmic examining device (i.e., an ophthalmoscope), which is similar to the instrument 100 or other, and includes a contained illumination system 211 as well as an imaging system 212. With regard to the illumination system 211, there are included separately controllable light sources 230, 231, a condensing lens 232 and a mirror 234 each disposed along a defined illumination axis 235. The light sources 230, 231 can be any generic light source, such as a filament based lamp, a metal halide lamp, a Xenon lamp, the end face of a fiber optic cable, a laser diode, or a single or multiple LED array.

In the herein depicted version, the light sources 230, 231 comprise single or multiple LED elements which can be illuminated individually or simultaneously. Exemplary LED light sources may comprise a source of white light such as RGB LEDs having wavelengths of the red (R), the green (G) and the blue (B) colors of the visible spectrum. A filter 233, such as an infrared filter for permitting light wavelengths of about 780-820 nanometers to pass therethrough or an amber light to permit light wavelengths of about 580-610 nanometers to pass through for enhancing eye examination in this instance. Either or both of the light sources 230, 231 may include a filter positioned distally of the light source. The light sources 230, 231 may further include aperture wheels 221, 225, respectively, to direct light, represented by light cones 226, 227, along the illumination axis 235 toward the retina of a patient. Each of the foregoing components are disposed within the lower handle portion 244 of the instrument head wherein the light sources 230, 231 are powered by contained batteries (not shown) as controlled by a processor (not shown).

The condenser lens 232, centered on the illumination axis 235, converges light from either of the light sources 230, 231 onto the mirror 234, which reflects the illuminating light along an imaging axis 222 to an objective lens 214, which causes the light to converge at an apex at or near the cornea of a patient's eye (not shown) and wherein the light diverges inside the patient's eye to illuminate a wide portion of the retina for viewing using the imaging system 212. Details relating to the herein described instrument are described in greater detail in U.S. Pat. No. 8,944,596, the entire contents of which are incorporated in their entirety.

Still referring to FIG. 3, the imaging system 212 includes at least one objective lens 214 (which also forms part of the illumination system 211) and at least one set of imaging lenses 218, each disposed within the instrument head 229 and aligned with the cover plate (window) 121 and the electronic imager 123 of the attached electronic device 120 along the defined imaging axis 222, including an electronic focus control of the camera of the supported electronic imaging device 120. Only a portion of an adapter/holding fixture 140 is shown in this view for clarity.

When attached and while the integrated electronic imaging element 123 of the attached electronic device 120 is aligned with the optical or imaging axis 222 of the herein described instrument 100/200 in order to receive images from the contained optical assembly 140, the flash output shown schematically as 127 in FIG. 3, of the portable electronic device 120, such as, for example, a portable iOS or Android® device, is not aligned with the aligned imaging axis 222 or illumination axis 235.

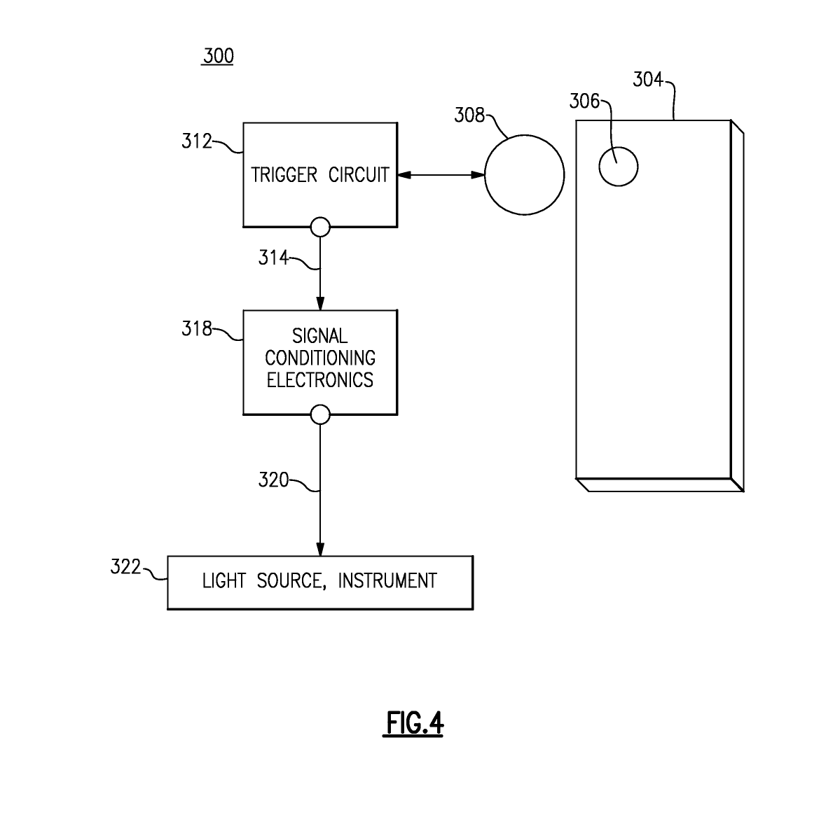

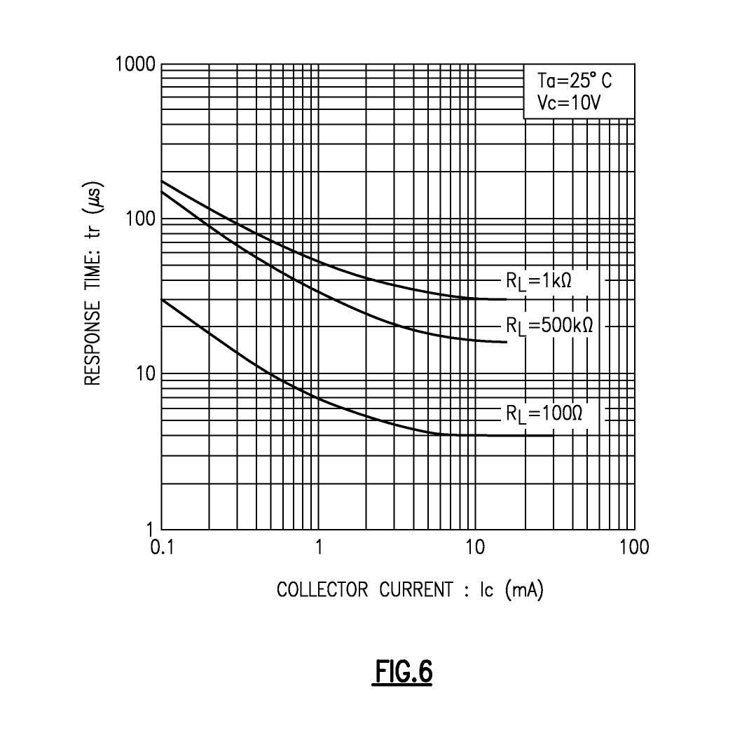

To tend to this issue and referring to FIG. 4, there is shown a high-level functional diagram of the auxiliary flash assembly 300 in accordance with an exemplary embodiment. Specifically, the flash output port or flash window of an attached portable electronic device 304 such as a smartphone, (e.g., iphone® or an Android®-based phone) when placed in the holding fixture 140, FIG. 2, of the instrument housing is aligned optically with a light detector 308, such as, for example, a photocell or photodetector. When a flash output is emitted by the portable electronic device 304 in response to a camera control on the display of the device, the output of the light detector 308 is received as an electrical signal by a trigger circuit 312, which is configured to release a trigger pulse, shown schematically as 314 in a synchronous manner. With reference to FIG. 6, it is shown that the response time of a photodetector used as a light detector can be on the order of 10-100 microseconds for an emitted light pulse (flash output). This response time is an order of magnitude faster than the typical 10 to 100 millisecond response time of the attached portable device.

Referring back to FIG. 4, the trigger pulse 314 is directed to signal conditioning electronics 318, shown schematically in this figure, which may include pulse shaping and amplifier circuits wherein the latter circuitry can be suitably configured in order to adjust the intensity, light dispersion and color temperature of at the least one contained light source, such as an LED, of the medical instrument to best suit the images that are being taken by the medical instrument, for example, taking into account issues that relate to an intended target of interest (e.g., eye, ear, nose, throat, skin, etc).

With the preceding high-level background, an exemplary electrical schematic diagram is provided in which a photodetector is connected to a battery 340 of the diagnostic instrument. The photodetector, which is shown here schematically as a photoresistor 344 is configured and aligned to detect light from the external flash 127, FIG. 3, of the attached electronic device 120, FIG. 3. Again, with reference to FIG. 6, it is evident that the receipt is synchronous or at least substantially synchronous with the emitted flash output. The output of the photoresistor 344 is directed through resistors 348 and 352 to respective field effect devices 356 and 360 that turn on corresponding LEDs 364, 368 for the duration of the trigger pulse. Resistors 372 and 376 control current (and therefore brightness) of the LEDs 364, 368. It should be noted that these resistors can be different in order to vary ratio between the LEDs 364, 368. According to this specifically described version, one of the LEDs 364 produces white light, while the remaining LED 368 produces an amber light in a manner analogous to those previously shown in FIG. 3 and thereby creating a multi-spectral flash according to this described implementation. According to this version, the amber LED 368 is configured to be in an “on state” constantly at a lower level as set by a potentiometer 380 and resistor 384. The foregoing circuitry assists in aiming the instrument relative to the target of interest (i.e., retina). For example and in the specific ophthalmic application, the human eye is less sensitive to amber illumination and the iris is less apt to close down. The flash trigger is configured to override this continuous operation and flashes the amber LED 368 at the higher level set by the resistor 376 during the trigger pulse.

In terms of operation and because the photocell (photoresistor 344) converts light into current and also provides current amplification, a separate amplifier is not required in this specific embodiment for the trigger pulse. In this version, the time duration of the boost flash is required to be the same as that of the flash of the external device 120. If this were not the case and depending on the application and intended medical target of interest for viewing, however, a pulse shaping circuit could be optionally inserted to provide the desired timing and duration of the boost (medical device) flash.

In this specific instrument, a set of aiming LEDs 400, 408 are additionally provided, such as red LEDs, that are ordinarily provided on part of the ophthalmic instrument 200. An example of such is described in U.S. Pat. No. 8,944,596B2, previously incorporated by reference, as well as numerous fundus cameras. The aiming LEDs 400, 408 are enabled by a switch 408 wherein the LEDs 400, 408 are caused to be inoperative during flash operation using resistor 412, but are ordinarily enabled for use. These aiming LEDs 400, 408 are configured to provide pre-flash illumination capability to initially position the instrument relative to a target of interest that is ordinarily difficult to align, such as the ear canal in the case of an otoscope, or the eye when using an eye cup on an ophthalmoscope.

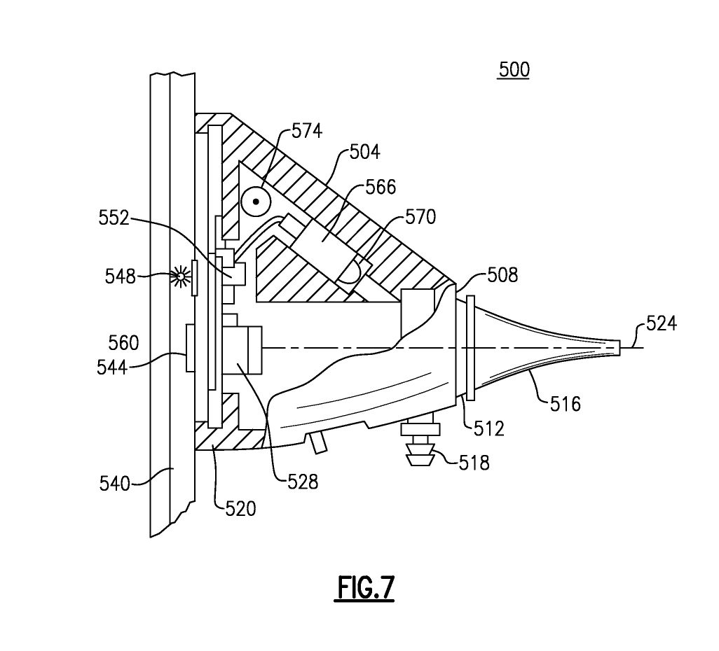

As noted, the herein described concepts can be applied to literally any medical diagnostic instrument having an attached portable device having an image-capture and flash (camera) capability. With reference to FIG. 7, an otoscopic instrument 500 is depicted. Only a portion of the instrument head 504 is shown; for example, the handle portion of the instrument 500 is not shown in this view. The instrument head 504 is defined by a distal end 508 having a frusto-conical insertion portion 512 onto which a hollow speculum tip element 516 is placed in overlaying relation. The interior of the instrument head 504 is substantially hollow, including an insufflation port 518 and with an opposing proximal end 520 being aligned with respective openings in the insertion portion 512 and tip element 516 along an optical (imaging) axis 524 to enable visualization of the ear canal (not shown) and more specifically, the tympanic membrane (eardrum) of a patient.

The otoscope 500 includes an optical (imaging) system that includes an objective lens (not shown), as well as a set of imaging lenses 528 disposed adjacent the proximal end 520 of the instrument head 504 and aligned along the axis 524 that are arranged to focus in relation to the imaging system (cover lens) of an attached portable electronic device, shown schematically as 540. The portable electronic device 540 according to this version is a smart-phone, although as previously discussed any appropriate mobile communications device can be utilized that includes an image-capture (electronic) device 544 and a source of flash illumination, shown schematically as 548. The device 540 can be releasably attached to the proximal end of the instrument head 504, using an adapter such as described in U.S. Patent Application Publication No. 2012/0320340 and U.S. Patent Application Publication No. 2013/0083185, each previously incorporated by reference herein. When attached, the source of flash illumination 548 is aligned with a light detector 552, such as a photocell, provided in the proximal end 520 of the instrument head 504 and the electronic (image capture) device 544 is aligned with the imaging axis 524.

The instrument 500 further includes signal conditioning electronics disposed on a circuit board 560 along with the trigger circuit, such as previously discussed and shown in FIG. 6, with a light source being provided in the form of an LED assembly 566 having an optical interface 570 disposed in an upper portion of the instrument head 504. A battery 574 can be provided in the instrument head 504 that is configured to provide power to the contained instrument components and circuitry.

In use, and when lighting conditions require the use of the source of flash illumination 548 for image capture using the device 548, the flash output is detected by the light detector 552, which is configured via the signal conditioning electronics and trigger circuit to produce a trigger pulse having a duration equal to that of the flash output and energizing the LED assembly 566 relative to the target of interest in which sufficient light is emitted through the attached speculum tip element that is reflected from the target of interest to the image-capture device 544.

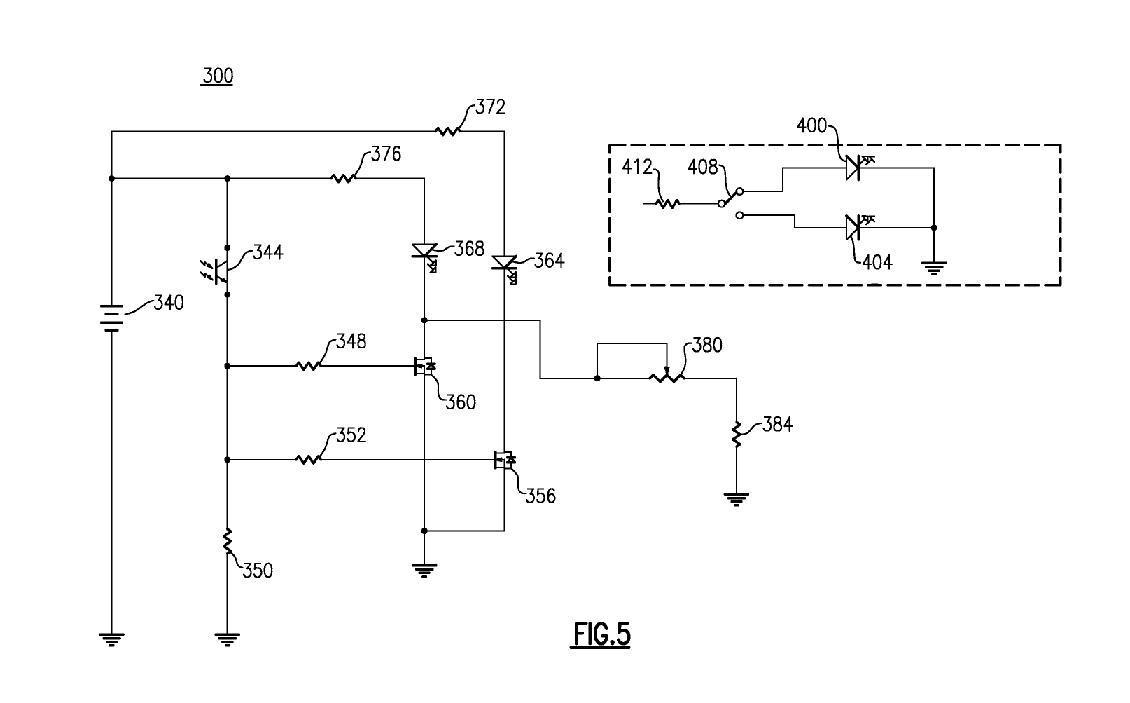

### PARTS LIST FOR FIGS. 1-7

100 medical diagnostic instrument
104 housing, instrument
108 distal end
109 eye cup
112 proximal end
116 handle portion
120 portable electronic device
121 cover plate
123 imager
127 flash output, portable electronic device
140 holding fixture
146 peripheral (side) rails
148 lower support
150 display, portable electronic device
154 user interface
200 medical diagnostic instrument
211 illumination system
212 imaging system
214 objective lens
218 imaging lenses
221 aperture wheel
222 imaging axis
225 aperture wheel
226 light cone
227 light cone
229 instrument head
230 light source
231 light source
232 condenser lens
233 filter
234 mirror
235 illumination axis
241 beamsplitter
244 lower handle portion, instrument
300 auxiliary flash apparatus
304 portable electronic device
306 flash assembly
308 light detector assembly
312 trigger circuit
314 trigger pulse
318 signal conditioning electronics
320 signal
322 light source, medical instrument
340 battery
344 photoresistor
348 resistor
350 resistor
352 resistor
356 field effect device
360 field effect device
364 LED, white
368 LED, amber
372 resistor
376 resistor
380 pot
384 resistor
400 LED, aiming
404 LED, aiming
408 switch
412 resistor
500 medical instrument
504 instrument head
508 distal end
512 insertion portion
516 speculum tip element
518 insufflation port
520 proximal end
524 optical (imaging) axis
528 imaging lenses
540 portable electronic device
544 imager, electronic
548 source of flash illumination
552 light detector
560 circuit board
566 light source
570 optical interface
574 battery

It should be readily apparent to one of average skill in the field that there are numerous variations and modifications that encompass the inventive concepts that are described herein and in accordance with the following claims:

1. An auxiliary flash apparatus for a medical diagnostic instrument having an attached portable electronic device, the portable electronic device having a source of flash illumination and an electronic imaging device, the auxiliary flash apparatus comprising:
a light detector configured to receive the flash output of the source of flash illumination;
a trigger circuit connected to the light detector;
signal conditioning electronics associated with the trigger circuit for receiving a trigger pulse from the trigger circuit synchronously or substantially synchronously with the flash output; and
in which the signal conditioning electronics is associated with a light source of the medical diagnostic instrument wherein the signal conditioning electronics are configured to produce a substantially synchronized and re-created flash using the light source.

2. The auxiliary flash apparatus as recited in claim 1, wherein the light source comprises at least one LED.

3. The auxiliary flash apparatus as recited in claim 1, wherein the signal conditioning electronics comprise a pulse shaping and amplifier circuit.

4. The auxiliary flash apparatus as recited in claim 1, wherein the electronic imaging device is at least one of a tablet PC or a smart-phone.

5. The auxiliary flash apparatus as recited in claim 1, wherein the light detector comprises a photocell.

6. A medical diagnostic instrument comprising:
an instrument housing having a light source;
a portable electronic device coupled to the instrument housing, the portable electronic device including an electronic imaging element and a source of flash illumination assembly:
a light detector configured to detect a flash output of the source of flash illumination;
a trigger circuit associated with the light detector;
signal conditioning electronics associated with the trigger circuit for receiving a trigger pulse from the trigger circuit synchronously or substantially synchronously with the flash output; and
in which the signal conditioning electronics are associated with a light source of the medical diagnostic instrument wherein the signal conditioning electronics are configured to produce a substantially synchronized and re-created flash using the light source.

7. The medical diagnostic instrument as recited in claim 6, wherein the light source comprises at least one LED.

8. The medical diagnostic instrument as recited in claim 6, wherein the signal conditioning electronics comprise a pulse shaping and amplifier circuit.

9. The medical diagnostic instrument as recited in claim 6, wherein the portable electronic device is a smart-phone.

10. The medical diagnostic instrument as recited in claim 6, wherein the light detector comprises a photocell.

11. The medical diagnostic instrument as recited in claim 6, wherein the medical diagnostic instrument is an ophthalmoscope.

12. A method for synchronously or substantially synchronously re-creating a flash of a portable electronic device that is attached to a medical instrument, the medical instrument including a light detector disposed in relation to an integrated flash assembly of the attached portable electronic device, the method comprising:
capturing an image of a target using the portable electronic device;
detecting light emitted by the flash assembly with a light sensing apparatus;
converting the light emitted by the flash assembly into an electrical signal;
directing the electrical signal to a trigger circuit and signal conditioning electronics synchronously or substantially synchronously with the flash output, and
configuring one or more light sources of the medical instrument to produce a substantially synchronized flash output.

13. The method as recited in claim 12, wherein the one or more light sources are further configured to optimize light intensity, dispersion and color temperature for an intended medical imaging application.

14. The method as recited in claim 12, in which the portable electronic device is at least one of a smart-phone or a tablet computer.

15. The method as recited in claim 12, wherein the light detector comprises a photocell.

16. The method as recited in claim 12, wherein the signal conditioning electronics comprise a pulse shaper and an amplifier circuit.

17. The method as recited in claim 12, wherein the medical instrument is an ophthalmoscope.

18. The method as recited in claim 12, wherein the one or more light sources are further configured to optimize light intensity, dispersion and color temperature for the purpose of aiming the instrument.

19. The method as recited in claim 12, in which a second set of one of more light sources is optimized for image capture in substantial synchronicity with the flash assembly.
